(19) United States
(12) Patent Application Publication
(10) Pub. No.: US 2021/0100325 A1
(43) Pub. Date: Apr. 8, 2021

(54) Necklace Inhaler Holder

(71) Applicant: Shirlette Satterwhite, Bowie, MD (US)

(72) Inventor: Shirlette Satterwhite, Bowie, MD (US)

(21) Appl. No.: 17/062,514

(22) Filed: Oct. 2, 2020

Related U.S. Application Data
(60) Provisional application No. 62/909,272, filed on Oct. 2, 2019.

(51) Int. Cl. A44C 15/00

(52) U.S. Cl. CPC A44C 15/003; A44C 15/005

(57) ABSTRACT

A necklace inhaler holder is shown and described. The necklace inhaler holder is comprised of a housing having a base and at least one flexible sidewall creating an interior volume. The at least one sidewall has an opening slit moving vertical to the base. An aperture located in the at least one sidewall adjacent to the base. A necklace strap is connected to the at least one sidewall.

### CROSS REFERENCE TO RELATED APPLICATIONS

This application claims the benefit of U.S. Provisional Application No. 62/909,272 filed on Oct. 2, 2019. The above identified patent application is herein incorporated by reference in its entirety to provide continuity of disclosure.

### BACKGROUND OF THE INVENTION

The present invention relates to carrying of inhalers. More particularly, the present invention provides a convenient necklace designed to hold an inhaler.

Many individuals have asthma or other conditions treated with inhalers. Often when a person has one of these conditions it is required that they have an inhaler with them at all times. In many cases this means that the inhaler will be left in a purse, backpack, car, or in a desk. This can make the inhaler difficult to locate and use if the need arises.

When having, for example, an asthma attack, even a few seconds may make a difference between recovering and going about your day and taking a trip to the hospital or worse. In some cases, when having an attack, it is necessary for another individual to retrieve the inhaler and help administer the necessary medication. If the inhaler is buried in a desk or other item, this may take an extended amount of time. These precious seconds may be saved if the inhalers were always kept on hand.

Consequently, there is a need for an improvement in the art of carrying, inhalers. The present invention substantially diverges in design elements from the known art while at the same time solves a problem many people face when having to quickly located an inhaler. In this regard the present invention substantially fulfills these needs.

### SUMMARY OF THE INVENTION

The present invention provides a necklace inhaler holder wherein the same can be utilized for providing convenience for the user having to carry and inhaler and find it in a timely manner. The necklace inhaler holder is comprised of a housing having a base and at least one flexible sidewall creating an interior volume. The at least one sidewall has an opening slit positioned vertically to the base. An aperture is located in the at least one sidewall adjacent to the base. A necklace strap is connected to the at least one sidewall.

Another object of the necklace inhaler holder is to have the necklace strap be an adjustable strap.

Another object of the necklace inhaler holder is to have the necklace strap be a removable strap.

Another object of the necklace inhaler holder is to have the necklace strap be removably secured to the at least one sidewall with a hook and loop fastener.

Another object of the necklace inhaler holder is to have a first window located in the at least one sidewall adjacent to the base and opposite the aperture

Another object of the necklace inhaler holder is to have the housing is configured to accept an inhaler therein.

Another object of the necklace inhaler holder is to have the opening slit be removably secured together using a hook and loop fastener.

Another object of the necklace inhaler holder is to have the opening slit be removably secured together using at least one snap fastener.

Other objects, features and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective rear view of an embodiment of the necklace inhaler holder.

FIG. 2 shows a perspective front view of an embodiment of the necklace inhaler holder.

FIG. 3 shows a perspective front view of an alternative embodiment of the necklace inhaler holder.

FIG. 4 shows a perspective front view of a further embodiment of the necklace inhaler holder.

### DETAILED DESCRIPTION OF THE INVENTION

Reference is made herein to the attached drawings. Like reference numerals are used throughout the drawings to depict like or similar elements of the necklace inhaler holder. For the purposes of presenting a brief and clear description of the present invention, a preferred embodiment will be discussed as used for the necklace inhaler holder. The figures are intended for representative purposes only and should not be considered to be limiting in any respect.

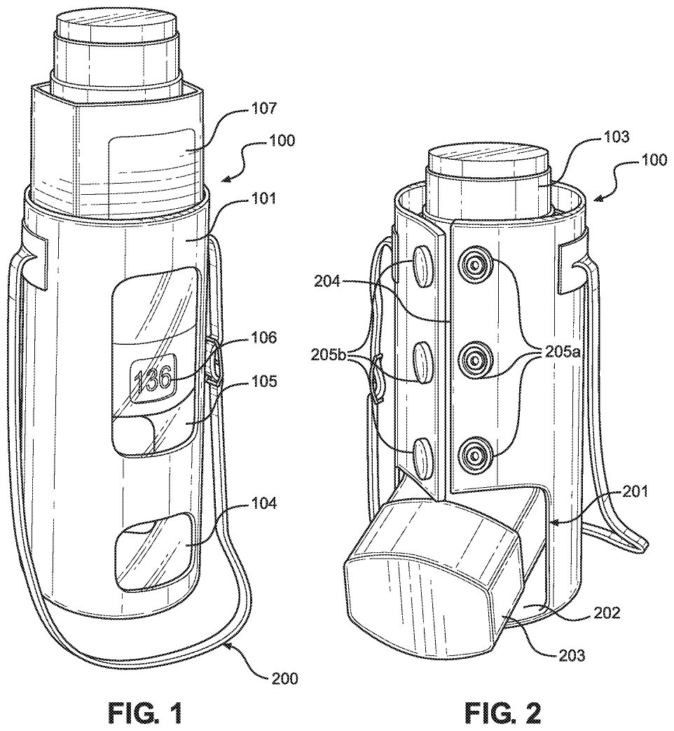

Referring now to FIG. 1, there is shown a perspective rear view of an embodiment of the necklace inhaler holder. The necklace inhaler holder is comprised of a housing 100 and a necklace strap 200. The carrier member 100 is comprised of a base having at least one sidewall 101 rising therefrom. In one embodiment the at least one sidewall 101 is comprised of rubber. In another embodiment the at least one sidewall 101 is comprised of plastic. In yet another embodiment the at least one sidewall 101 is made from silicone.

The at least one sidewall 101 has an open top end 102. The open top end 10, leads to an interior volume. The open top end 102 and the interior volume are configured to accept an inhaler 103 therein. In one embodiment the inhaler 103 will fit flush against the base of the carrier member 100. In another embodiment the base is flat and the inhaler 103 will have a gap between the base of the carrier member 100 and the bottom of the inhaler 103. In one embodiment sidewall 101 is dimensioned such that the inhaler 103 protrudes through the open top end 102. In another embodiment the inhaler 103 is encapsulated by the at least one sidewall 101.

In various embodiments the at least one sidewall 101 has at least one window therein. In one embodiment the at least one window is simply an aperture through the sidewall 101. In another embodiment the at least one window has a protective covering located thereon. In one embodiment the covering is transparent material to allow for visualizing the interior volume. In the shown embodiment there is a lower window 104 and an upper window 105. The lower window 104 is positioned adjacent to the base. In one embodiment the lower window 105 will align with a dosage number 106 displayed on the inhaler 103. The upper window 105 is positioned above the lower window 104. In one embodiment the upper window 105 will align with the prescription information 107 located one the inhaler 103.

Referring now to FIG. 2, there is shown a perspective front view of an embodiment of the necklace inhaler holder. There is a mouthpiece aperture 201 located in the at least one sidewall 101. The mouthpiece aperture 201 is located opposite the windows as described above in FIG. 1. The mouthpiece aperture 201 is placed adjacent to the base 202. The mouthpiece aperture 201 is configured to accept the mouthpiece 203 of the inhaler 103 therethrough. This will allow the inhaler 103 to be used while still located within the housing 100.

In some embodiments there is an opening slit 204 in the at least one sidewall. In one embodiment the opening slit 204 is vertical in relation to the base 202. In one embodiment the opening slit 204 is connected to the mouthpiece aperture 201. The opening slit 204 will allow for easier placement of the inhaler 203 within the housing 100.

In various embodiment the opening slit 204 is configured to be operably secured together. In these embodiments the at least one sidewall is elongated at the opening slit 204 such that each side of the opening slit 204 will overlap. This means that the opening slit 204 can be held together to hold the inhaler 103 in place and opened for inserting or removing the inhaler 103. In the shown embodiment the opening slit 204 is securable with a plurality of snaps 205a, 205b. In the shown embodiment a first part of the snap 205a is located on one side of the opening slit 204. A second part of the snap 205b is located on the opposite side of the opening slit 204.

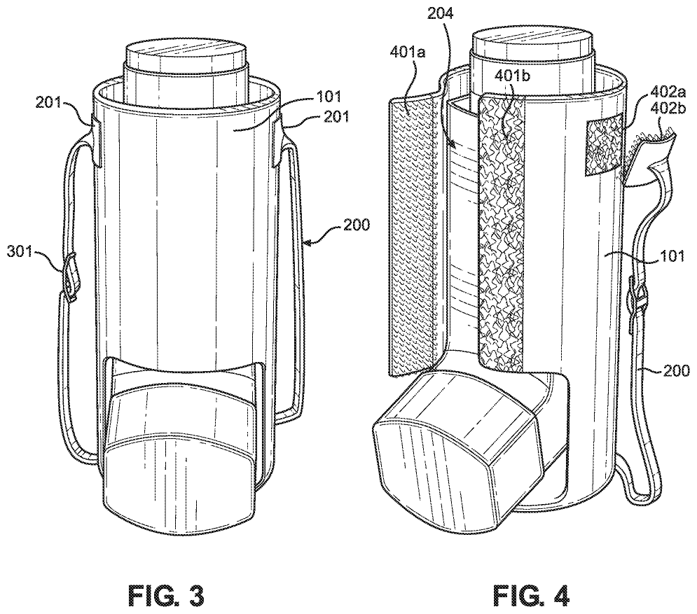

Referring now to FIG. 3, there is shown a perspective front view of an alternative embodiment of the necklace inhaler holder. In the shown embodiment there is no opening slit within the at least one sidewall 101. In this embodiment the housing 100 is comprised of material that has the ability to stretch. The material will expand to allow the inhaler 103 to be fitted within the housing. The material will then resize holding the inhaler 103 in place.

The necklace inhaler holder is further comprised of a necklace strap 200. The necklace strap 200 is connected to the at least one sidewall 101. In the shown embodiment the necklace strap 200 is connected to the at least one sidewall 101 in two places 201. In other embodiments the necklace strap 200 is connected to the at least one sidewall 101 in only one location. In the shown embodiment the necklace strap 200 is permanently connected to the at least one sidewall 101. In other embodiments as described below the necklace strap 200 is removably secured to the at least one sidewall 101.

In some embodiments the necklace strap 200 has a fixed length. In other embodiments the necklace strap 200 has an adjustable length. In one embodiment the necklace strap 200 has an adjuster 301 located thereon. In the shown embodiment the adjuster 301 will allow part of the necklace strap 200 to be pulled through the adjuster 301 and the length of the necklace strap 200 will be adjusted. In another embodiment the necklace strap 200 is a two-part strap. This will allow one part of the necklace strap 200 to be pulled through the adjuster 301. This will adjust the length of the necklace strap 200.

Referring now to FIG. 4, there is shown a perspective front view of a further embodiment of the necklace inhaler holder. In the shown embodiment there is an alternative way shown to close the opening slit 204. In the shown embodiment a hook and loop fastener 401a, 401b is used. In this embodiment one part of the hook and loop fastener 401b is located on the backside of the at least one sidewall 101 on one side of the opening slit 204. A second part of the hook and loop fastener 401a is located on the front of the at least one sidewall 101 of the opposite side of the opening slit 204.

Further, in the shown embodiment the necklace strap 200 is removably connected to the at least one sidewall 101. In the shown embodiment a hook and loop fastener 402a, 402b is used to secure the necklace strap 200 to the at least one sidewall 101. In this embodiment a first part 402a of the hook and loop fastener is secured to the at least one sidewall 101. A second part 402b of the hook and loop fastener is secure to the necklace strap 200. This will allow the necklace inhaler holder to be easily removed. In some instances, this will allow the inhaler necklace to be ripped from the neck of the wearer preventing harm to the wearer.

In other embodiments the necklace strap 200 is removably connected using other methods. In one embodiment the necklace strap 200 is connected using a snap. In another embodiment the necklace strap 200 is connected using a connector. In yet another embodiment the necklace strap 200 is attached using a hole and pin method. In this method the pin will have an expanded head that contract to fit through the hole then expand once through removably holding the necklace strap 200 to the at least one sidewall 101.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A necklace inhaler holder, the necklace comprising:
a housing having a base and at least one sidewall creating an interior volume;
an aperture located in the at least one sidewall adjacent to the base;
a necklace strap secured to the at least one sidewall.

2) The necklace inhaler holder of claim 1, wherein the necklace strap is an adjustable strap.

3) The necklace inhaler holder of claim 1, wherein the necklace strap is removably secured to the at least one sidewall.

4) The necklace inhaler holder of claim 3, wherein the necklace strap is removably secured to the at least one sidewall with a hook and loop fastener.

5) The necklace inhaler holder of claim 1, further comprising a first window located in the at least one sidewall adjacent to the base and opposite the aperture

6) The necklace inhaler holder of claim 1, wherein the housing is configured to accept an inhaler therein.

7) A necklace inhaler holder, the necklace comprising:
a housing having a base and at least one flexible sidewall creating an interior volume;
the at least one sidewall has an opening slit moving vertical to the base;
an aperture located in the at least one sidewall adjacent to the base;
a necklace strap is secured to the at least one sidewall.

8) The necklace inhaler holder of claim 7, wherein the necklace strap is an adjustable strap.

9) The necklace inhaler holder of claim 7, wherein the necklace strap is removably secured to the at least one sidewall.

10) The necklace inhaler holder of claim 9, wherein the necklace strap is removably secured to the at least one sidewall with a hook and loop fastener.

11) The necklace inhaler holder of claim 7, further comprising a first window located in the at least one sidewall adjacent to the base and opposite the aperture

12) The necklace inhaler holder of claim 7, wherein the housing is configured to accept an inhaler therein.

13) The necklace inhaler holder of claim 7, wherein the opening slit is removably secured together using a hook and loop fastener.

14) The necklace inhaler holder of claim 7, wherein the opening slit is removably secured together using at least one snap fastener.
